(19) United States
(12) Patent Application Publication
(10) Pub. No.: US 2026/0232469 A1
(43) Pub. Date: Aug. 13, 2026

(54) STENT DEVICE CAPABLE OF INJECTING DRUG

(71) Applicant: TAEWOONG MEDICAL CO., LTD., Gyeonggi-do (KR)

(72) Inventors: Sang Min JOO, Sejong (KR); Yeo Jin JUN, Sejong (KR)

(21) Appl. No.: 19/157,701

(22) PCT Filed: Feb. 26, 2024
(86) PCT No.: PCT/KR2024/095368

§ 371 Date: Aug. 19, 2025

(30) Foreign Application Priority Data
Mar. 14, 2023 (KR) 10-2023-0033331

(51) Int. Cl. A61F 2/966; A61F 2/91; A61F 2/95; A61M 25/00

(52) U.S. Cl. CPC A61F 2/966; A61F 2/91; A61F 2/9522; A61M 25/0069; A61M 25/0097; A61F 2250/0067; A61M 2025/0004

(57) ABSTRACT

A stent device capable of injecting a drug, according to an embodiment of the present disclosure, is a stent device having a stent coupled to a catheter. The stent device comprises: a porous thin-film stent capable of storing a drug to be supplied to a point at which the stent procedure is performed, and a catheter to which the stent is coupled and which has a drug inlet part formed on the body thereof to allow the separate supply of the drug during the stent procedure, thereby enabling the injection of the drug before the stent procedure, followed performing the stent procedure.

### TECHNICAL FIELD

The present disclosure relates to a stent system capable of selectively injecting a drug and, more particularly, to a stent device including a drug stent that enables a user to select and use a drug during a stent procedure and a catheter used therefor.

### BACKGROUND ART

A stent is a tubular artificial device inserted into the human body to resolve blockages in vascular and non-vascular tubular tissues and is used for the purpose of increasing patient convenience and treating localized diseases. The demand for stents continues to grow worldwide.

Recently, drug-eluting stents (DES) coated with various drugs have been developed to improve the therapeutic efficacy of stents. The drug-eluting stent can effectively and continuously deliver a topical drug and significantly reduce the rate of restenosis in the stent, according to the report.

The drug-eluting stent, which was initially developed, was a system that only had the drug coated thereon. However, due to the weak coating film, the drug was rapidly released in large amounts at the beginning of use, and the restenosis prevention performance and treatment effect were low. Therefore, to solve the problem, drug-eluting coatings using polymers are currently being developed and are gaining traction in the stent market.

In this concept, the drug-eluting stents being developed using polymers are manufactured in three representative forms. First, in a monolithic device form, a drug is contained within a polymeric matrix, and release of the drug is controlled by the rate of diffusion of the polymeric matrix (a representative example is Boston Scientific's “Taxus” containing paclitaxel). In a reservoir system (a representative example is Johnson & Johnson's “Cypher”, which contains sirolimus), a drug is contained in a central polymer matrix and surrounded by a thin polymer membrane on the outer surface, and drug release is controlled by the rate of diffusion of the drug across the polymer membrane. The drug-eluting stent of the drug-eluting films is surrounded by a polymeric film that encases a drug-loaded polymeric coating across its surface.

However, the use of the drug-injected stents is limited because the stent itself is distributed with the drug injected and only one selected drug is used through it.

The matters described above as background technology are intended to provide a better understanding of the context of the present disclosure and are not intended to constitute a disclaimer of the prior art known to those of ordinary skill in the art.

### DOCUMENT OF RELATED ART

### Patent Document

Korean Patent Application Publication No. 10-2015-0061949 (Jun. 5, 2015)

### DISCLOSURE

### Technical Problem

The present disclosure is provided to solve the above problem, and an objective of the present disclosure is to provide a stent device capable of selecting a drug while enabling a user to select a drug for a stent procedure and inject the drug through a catheter.

### Technical Solution

In order to achieve the above objective, according to an embodiment of the present disclosure, there is provided a stent device capable of injecting a drug, and the stent device, in which a stent is coupled to a catheter, includes: the stent made of a porous thin film and capable of storing a drug to be supplied to a position where a stent procedure is performed, and the catheter to which the stent is coupled, the catheter including a drug inlet part provided in a main body to separately supply the drug during the stent procedure, thereby performing the stent procedure by injecting the drug before the procedure.

The catheter may include: the main body in which an inner passage is formed; a wire passing through the main body with the stent installed therein; a stent-loading part formed around the wire and holding the stent mounted thereto; a tip connected to the stent-loading part and directed to an internal part of human body; and the drug inlet part provided on a side surface of the main body and configured to inject the drug into an internal space.

The drug inlet part may be formed in a diagonal direction of the main body and has a Y-shape.

The catheter may include an outer shaft at the stent-loading part to be brought into contact with the tip according to a length thereof to prevent leakage of the drug supplied into the internal space.

The porous thin film of the stent may have pores with a size of less than or equal to 500 μm.

The thickness of the porous thin film of the stent may be less than or equal to 100 μm.

### Advantageous Effects

According to the present disclosure, the stent device capable of injecting a drug has the following effects.

First, the stent device can directly supply drugs in many injectable forms, which are already available, into a location in the human body where the stent is used, thereby overcoming the limitations of existing injections.

Second, unlike an existing method of directly coating a stent with a drug when manufacturing stents, the stent device is configured to enable an operator of a stent procedure to select and inject a user-required drug for the stent procedure, a drug can be selected and injected by the operator, and the drug suitable for treatment of a lesion can be absorbed into a location where the stent is used.

### DESCRIPTION OF DRAWINGS

FIG. 1 is a view showing a stent device according to an embodiment of the present disclosure.

FIG. 2 is an enlarged view showing a drug inlet part of the stent device according to the embodiment of the present disclosure.

FIG. 3 is an enlarged view showing a stent-loading part of the stent device according to the embodiment of the present disclosure.

FIG. 4 is a view showing a stent of the stent device according to the embodiment of the present disclosure.

FIG. 5 is an enlarged view showing the drug inlet part in a process of injecting a drug into the stent device according to the embodiment of the present disclosure.

FIG. 6 is an enlarged view showing the stent-loading part in a process of injecting a drug into the stent device according to the embodiment of the present disclosure.

FIG. 7 is a view showing the stent of the stent device into which a drug is injected according to the embodiment of the present disclosure.

### MODE FOR INVENTION

Terms used herein are selected to describe embodiments and thus should not be construed as the limit of the present invention. An element expressed in a singular form in this specification may be plural elements unless it is necessarily singular in the context. The terms “comprise” and/or “comprising” means inclusion of a shape, number, process, operations, member, element, and/or a group of those, but do not mean exclusion of or denial of addition of another shape, number, process, operation, element, and/or a group of those.

In the flowing description, unless otherwise defined, all terms including technical and scientific terms used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinbelow, a stent device according to an exemplary embodiment of the present disclosure will be described with reference to accompanying drawings.

According to the embodiment of the present disclosure, a stent device, in which a stent is coupled to a catheter, includes: the stent of a porous thin film capable of storing a drug to be supplied to a location where a stent procedure is performed, and the catheter coupled with the stent and including a drug supply part, the drug supply being formed at a main body to separately supply a drug during the stent procedure and supplied with the drug before the stent procedure to perform the stent procedure.

In other words, the stent device includes a stent made of a porous thin film to absorb the drug and has a form that may inject an operator-selected drug into the stent. This leads to an innovative effect of directly injecting a drug with the catheter from an existing drug stent having a stent to which a manufacturer-selected drug is applied.

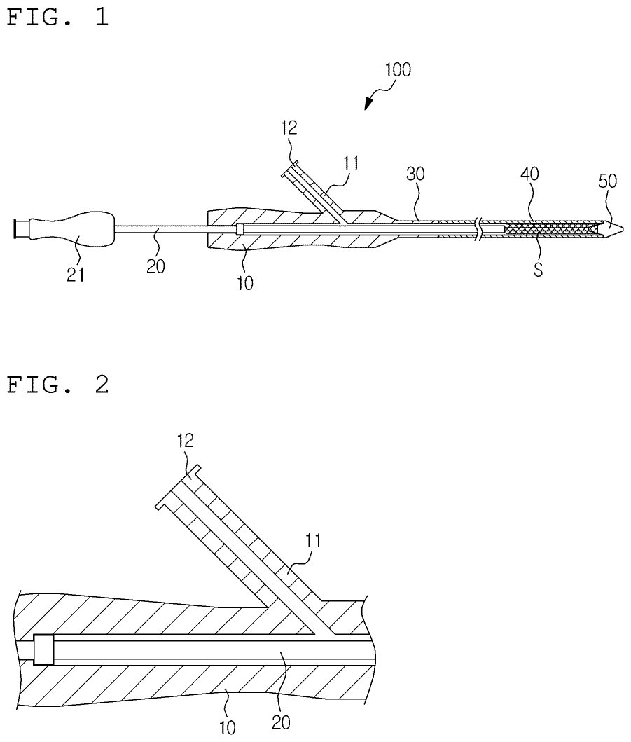

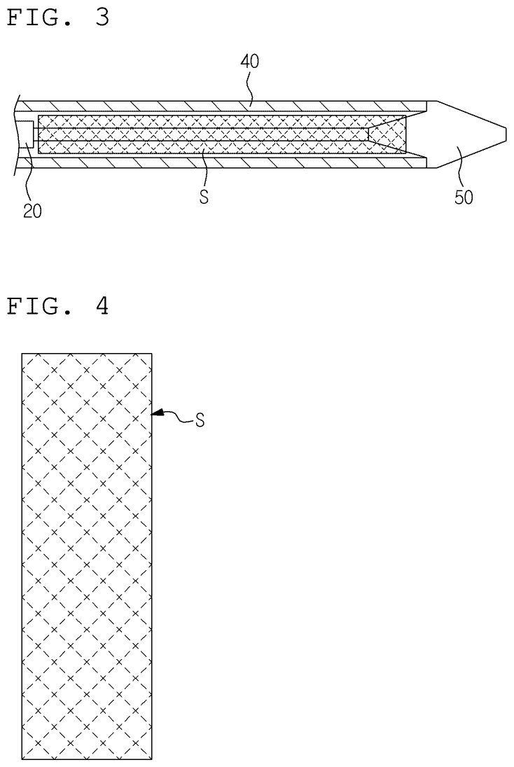

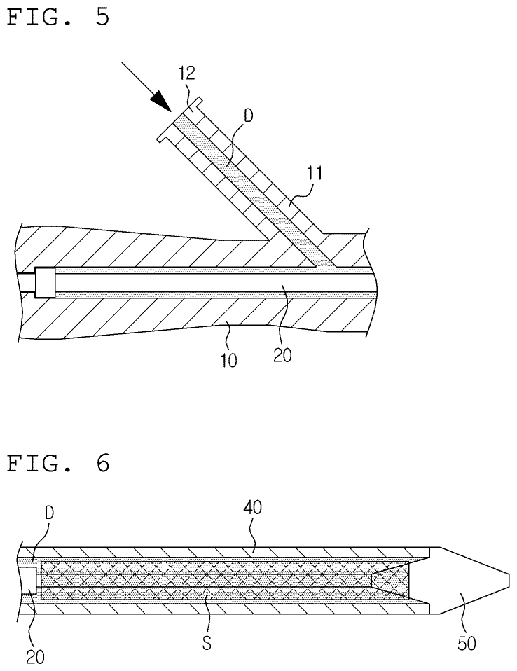

FIG. 1 is a view showing a stent device according to the embodiment of the present disclosure. FIG. 2 is an enlarged view showing a drug inlet part of the stent device according to the embodiment of the present disclosure. FIG. 3 is an enlarged view showing a stent-loading part of the stent device according to the embodiment of the present disclosure. FIG. 4 is a view showing a stent of the stent device according to the embodiment of the present disclosure.

Referring to FIGS. 1 to 4, according to the embodiment of the present disclosure, a stent device 100 includes a main body 10, a wire 20, a tip 50, a drug inlet part, and a stent S.

The main body 10 has a cylindrical shape with an internal space and open opposite ends. The internal space of the main body includes the wire 20, the wire being movable to allow the stent S to be mounted to a diseased part. A first end of the wire 20 may be coupled to a pusher 21 serving as a handle, and a second end of the wire 20 may be coupled to the tip 50 that serves to guide the stent when the stent accesses the diseased part.

The drug inlet part is formed on a side surface of the main body 20 to inject a drug into the internal space. The drug inlet part has any shape as long as it can supply a drug into the space where the stent S is mounted to the wire 20 in the internal space of the main body 10, from the side surface of the main body 10. For example, a method of forming a hole in the side surface and injecting a drug, a method of forming a separate passage in the side surface and injecting a drug, and a method of forming a packing with a shape capable of inserting a syringe in the side surface of the main body and injecting the drug by the syringe are conceivable. In the embodiment, a branch 11 is formed in a Y-shape to stably inject a drug, and an end of the branch is formed in a drug inlet 12 to inject a liquid drug by a syringe or directly inject the drug. In the embodiment, the shape of the branch is described as a Y shape, but this form is only to illustrate the formation of the branch 11 on the side surface of the main body 10, and the form of the branch is not limited to the shapes of alphabets from the description.

An end of the main body 10 is formed such that an outer shaft 30 is brought into contact with the tip before the procedure to prevent the drug inserted into the internal space from leaking to the external space. The length of the outer shaft 30 may be preferably formed by a length sufficient for the tip 50 to be brought into close contact with the end after the stent S is mounted to a stent-loading part 40. As formed above, the tip 50 may serve as a stopper and prevent the injected drug from leaking to the external space.

The stent S has a net shape made of wires to allow the injected drug to be absorbed, and a surface thereof is wrapped with a porous film. The porous film has a shape that may allow the drug to be released when the drug is injected, and should maintain this shape until a stent procedure is performed in a diseased part.

The porous thin film used herein is one selected from a group consisting of fluorine-based polymer, silk, cellulose acetate, nylon, polyethylene terephthalate (PET), polyurethanes (PU), silicon, and extracellular matrix natural polymer. The material selected herein should be selected in consideration of the amount of drug to be injected. The extracellular matrix natural polymer is a natural polymer that may be used as a major component of the extracellular matrix, which is a polymeric substance used as a tissue in charge of structural support of animals, and is mainly responsible for structural support in animals. For example, the extracellular matrix can be divided into connective tissue fibers (collagen, elastin, etc.), amorphous components (glycosaminoglycans, proteoglycans, and glycoproteins), and extracellular fluid. Preferably, the extracellular matrix may be collagen, hyaluronic acid, or gelatin.

The pore size of the porous thin film may be equal to or greater than 1 μm and equal to or less than 500 μm. A pore of the porous thin film should have a size sufficient to absorb the injected drug and maintain the amount of the injected drug or more. When the pore size is less than 1 μm, the drug may not be absorbed into the stent, and when the size is greater than 500 μm, the strength of the thin film may not be maintained, so the pore size equal to or greater than 1 μm and equal to or less than 500 μm is preferred.

The thickness of the porous thin film may be equal to or greater than 10 μm and equal to or less than 500 μm. The thickness of the porous thin film should be sufficient to absorb the injected drug and hold a certain amount of the injected drug or more. When the thickness is less than 100 μm, a sufficient amount of drug may not be absorbed, and when the thickness is greater than 500 μm, the stent may not move smoothly inside the catheter, so the thickness equal to or greater than 100 μm and equal to or less than 500 μm is preferred.

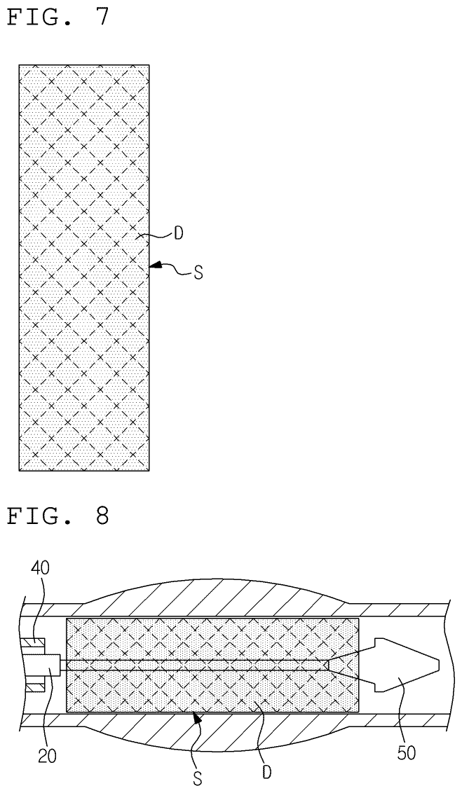

Hereinbelow, operation of the stent device according to the embodiment of the present disclosure will be described. FIG. 5 is an enlarged view showing the drug inlet part during a process of injecting a drug into the stent device according to the embodiment of the present disclosure. FIG. 6 is an enlarged view showing the stent-loading part during the process of injecting a drug into the stent device according to the embodiment of the present disclosure. FIG. 7 is a view showing a shape of a drug injected into the stent of the stent device according to the embodiment of the present disclosure.

Referring to FIGS. 5 to 7, first, the stent S is installed at the stent-loading part 40 through the tip 50. After installation, an end of the outer shaft 30 of the main body and the tip 50 are brought into close contact with each other, and then the drug D may be injected through the drug inlet 12. A sufficient amount of the drug D is supplied to the stent S, and then the procedure of the stent S is performed into a diseased part of a patient. The drug D absorbed in the stent S is directly supplied to the corresponding diseased part.

As described above, according to the embodiment of the present disclosure, the stent device can directly supply a drug in many injectable forms that are already available into a location in the human body where the stent is placed, thereby overcoming the limitations of existing injections. Furthermore, unlike the existing method of directly coating a stent with a drug during the manufacturing of stents, the stent device is configured to enable an operator of a stent procedure to select and inject a user-required drug for the stent procedure, and the operator can select a drug him/herself and inject the drug so that it is possible to absorb a drug suitable for treatment of a lesion into a location where the stent is used.

Although the preferred embodiment of the present disclosure has been described for illustrative purposes, and those skilled in the art will appreciate that various modifications, additions, and substitutions are possible, without departing from the scope of the present disclosure as disclosed in the accompanying claims.

As described above, the embodiment disclosed herein should be considered from an illustrative perspective and not a limiting perspective. The scope of the present disclosure is presented in the claims, not in the above-described description, and any differences within the scope of the claims have to be construed as included in the present disclosure.

[Description of Reference Numeral]

| | |
|---|---|
| 10: main body | 11: branch |
| 12: drug inlet | 20: wire |
| 21: pusher | 30, 40: outer shaft |
| 50: tip | S: stent |
| D: drug | |

FIG. 8 is a view showing an insertion of the stent into a diseased part, the stent being supplied with a drug by the stent device according to the embodiment of the present disclosure.

1. A stent device with a stent coupled to a catheter, the stent device comprising:
the stent made of a porous thin film and capable of storing a drug to be supplied to a location where a stent procedure is performed, and the catheter to which the stent is coupled, the catheter including a drug inlet part provided in a main body to separately supply the drug during the stent procedure, thereby performing the stent procedure by injecting the drug before the stent procedure.

2. The stent device of claim 1, wherein the catheter comprises:
the main body in which an inner passage is formed;
a wire passing through the main body with the stent installed therein;
a stent-loading part formed around the wire and holding the stent mounted thereto;
a tip connected to the stent-loading part and directed to an internal part of human body; and
the drug inlet part provided on a side surface of the main body and configured to inject the drug into an internal space.

3. The stent device of claim 2, wherein the drug inlet part is formed in a diagonal direction of the main body and has a Y-shape.

4. The stent device of claim 2, wherein the catheter includes an outer shaft at the stent-loading part to be brought into contact with the tip according to a length thereof to prevent leakage of the drug supplied into the internal space.

5. The stent device of claim 1, wherein the porous thin film of the stent has pores with a size of less than or equal to 500 μm.

6. The stent device of claim 1, wherein the thickness of the porous thin film of the stent is less than or equal to 500 μm.

7. The stent device of claim 1, wherein the porous thin film of the stent is one selected from a group consisting of fluorine-based polymer, silk, cellulose acetate, nylon, polyethylene terephthalate (PET), polyurethanes (PU), silicon, and extracellular matrix natural polymer.
